(19) United States
(12) Patent Application Publication
(10) Pub. No.: US 2018/0116853 A1
(43) Pub. Date: May 3, 2018

(54) THERAPY SOCK

(71) Applicant: Teresa Jones, Rockwall, TX (US)

(72) Inventor: Teresa Jones, Rockwall, TX (US)

(21) Appl. No.: 15/340,451

(22) Filed: Nov. 1, 2016

(51) Int. Cl. A61F 5/01; A61F 5/34

(52) U.S. Cl. CPC A61F 5/0111; A61F 5/34; A61F 5/019

(57) ABSTRACT

A therapy sock that includes: a high ankle support; an anterior ankle support; an arch support connecting to the anterior support; a ball support; a plurality of toe pockets leading from the ball support; and toe separators between each toe pocket. The toe separators are preferably composed of a gel substance. The high ankle support may include at least three reinforcement bands. The anterior ankle support includes two reinforcement bands leading from the high ankle support.

### BACKGROUND OF THE INVENTION

### Field of Invention

The present invention relates to a therapy sock used to support an individual's foot.

### Description of Related Art

Various socks include support features in addition to the basic function of covering an individual's foot. Support socks come in various varieties and can be helpful in supporting an individual's ankle, arch or heel. Further some toe socks have been developed that separate the toes to provide further comfort of the individual's foot. One particular problem individuals have is bunion pain that should be addressed to avoid problems. Improper alignment of an individual's foot, ankle or toes can transfer pain to an individual's hip, knees and even shoulder joints. Further proper alignment can help avoid injuries to an individual's ligaments while performing certain sporting activities. Also proper support of the foot can help correct foot posture and general posture of an individual. As a result, it is advantageous to have a proper supporting sock that addresses bunion support and possible bunion pain while wearing the sock.

### SUMMARY OF THE INVENTION

The present invention relates to a therapy sock that includes: a high ankle support; an anterior ankle support; an arch support connecting to the anterior support; a ball support; a plurality of toe pockets leading from the ball support; and toe separators between each toe pocket. The toe separators are preferably composed of a gel substance. The high ankle support may include at least three reinforcement bands. The anterior ankle support includes two reinforcement bands leading from the high ankle support.

### BRIEF DESCRIPTION OF DRAWINGS

FIG. 1 depicts a therapy sock in accordance with the present invention.

### DETAILED DESCRIPTION

The present invention relates to a therapy sock used to support an individual's foot and provide proper alignment thereof. This support therapy sock according to the present invention includes high ankle support connected to an anterior reinforcement that leads to the arch area of the foot. The arch area provides further reinforcement and support connecting to support on the undersigned of the foot at the beginning of the ball of the foot with toe pockets extending from the ball support. The toe pockets further include separators that separate each toe pocket therefore providing firm support for the foot from the toe back up into the arch onto the anterior of the ankle.

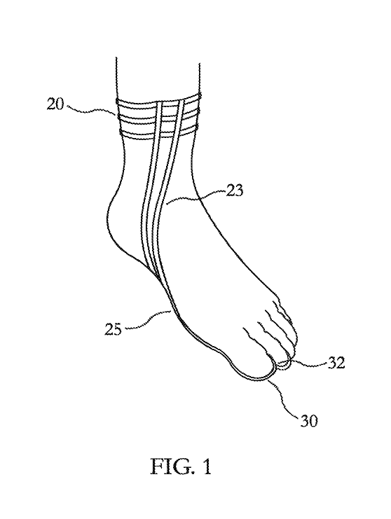

In reference to FIG. 1, a therapy sock in accordance with the present invention is depicted. This therapy sock includes a high ankle support 20 that wraps around the high ankle or just above the ankle once fitted onto an individual's foot. The sock further includes the anterior support reinforcements 23 leading from the high ankle support 20. The anterior support 23 leads into arch support 25. The support is created in the form of reinforcement bands where reinforcement bands are used at the high ankle support 20, as shown three bands are depicted. Two bands are provided for the anterior support 23 leading into the arch support 25. At the bottom of the foot a ball support (not shown) is provided that connects to toe pockets 30. Five toe pockets 30 are provided with a toe separator 32 between each toe pocket 30. Preferably, the toe pockets 30 are gel support with a gel used for the toe separators. The arch support 25 is preferably provided by using tight sturdy material to give proper arch support and to relieve bunion pain. The reinforcement bands are comprised of knit material that again provides firm support of the high ankle and anterior portion of the foot. This arrangement offers protection for the talofibular and calcaneofibular ligaments. The separators 32 help to provide ample space between each toe and therefore correcting muscle alignment and ligament development.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A therapy sock comprising:
a high ankle support;
an anterior ankle support;
an arch support connecting to the anterior support;
a ball support;
a plurality of toe pockets leading from the ball support; and
toe separators between each toe pocket.

2. The therapy sock according to claim 1, where the toe separators are composed of a gel substance.

3. The therapy sock according to claim 1, where the high ankle support includes at least three reinforcement bands.

4. The therapy sock according to claim 1, where in the anterior ankle support includes two reinforcement bands leading from the high ankle support.
